(19) United States
(12) Patent Application Publication
(10) Pub. No.: US 2004/0242670 A1
(43) Pub. Date: Dec. 2, 2004

(54) Process for preparation of amorphous atorvastatin calcium

(76) Inventors: Sonny Sebastian, Hyderabad (IN); Sitaramaiah Devarasetty, Hyderabad (IN); Ramesh Dandala, Hyderabad (IN); Meenakshisunderam Sivakumaran, Hyderabad (IN)

Correspondence Address: Jay R. Akhave, 845 Pomello Dr., Claremont, CA, 91711, US

(21) Appl. No.: 10/858,261

(22) Filed: Jun. 1, 2004

(30) Foreign Application Priority Data
Jun. 2, 2003 (IN) 438/MAS/2003
Jun. 2, 2003 (IN) 439/MAS/2003

(51) Int. Cl. A61K 31/401

(52) U.S. Cl. 514/423; 548/537

(57) ABSTRACT

Process for the preparation of amorphous form of Atorvastatin calcium of Formula I

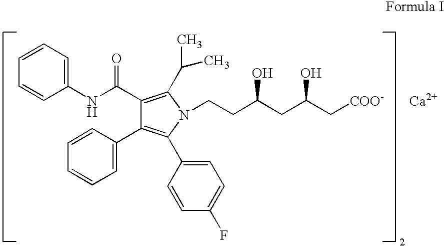

which comprises the conversion of the crystalline Atorvastatin calcium to amorphous Atorvastatin calcium.

### CROSS REFERENCE TO THE RELATED APPLICATION

This application claims priority from Indian Application Nos. 438/MAS/2003 and 439/MAS/2003, both filed on Jun. 2, 2003.

### FIELD OF THE INVENTION

The present invention relates to a process for the preparation of pure amorphous Atorvastatin calcium from crystalline atorvastatin calcium or from a mixture of crystalline and amorphous atorvastatin calcium by spray drying or by pulverization method.

### BACKGROUND OF THE INVENTION

Atorvastatin of Formula I is a selective inhibitor of the enzyme 3-hydroxy-3-methyl glutaryl CoA reductase (HMG-CoA reductase) and is chemically known as [R—(R*,R*)-2-(4-fluorophenyl)-β,δ-dihydroxy-5-(1-methylethyl)-3-phenyl-4-[(phenylamino)carbonyl]-1H-pyrrole-1-heptanoic acid hemi calcium salt. As such Atorvastatin calcium is a potent lipid-lowering compound and is thus useful as a hypocholesterolemic agent.

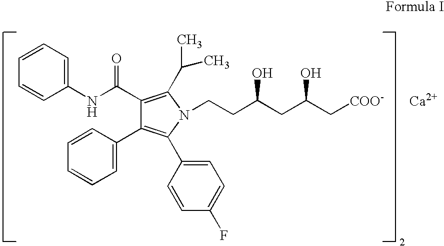

Atorvastatin was disclosed in U.S. Pat. No. 5,273,995 as ring opened dihydroxy acid form. U.S. Pat. Nos. 5,003,080; 5,097,045; 5,103,024; 5,124,482; 5,149,837; 5,155,251; 5,216,174; 5,245,047; 5,248,793; 5,280,126; 5,397,792 and 5,342,952, which are herein incorporated by reference, disclose various processes and key intermediates for preparing Atorvastatin calcium. The processes described in the above mentioned US patents do not give amorphous Atorvastatin calcium consistently but give a mixture of its crystalline and amorphous forms, which has unsuitable filtration and drying characteristics.

U.S. Pat. No. 6,087,511 describes the preparation of amorphous Atorvastatin calcium by dissolving the compound in a non-hydroxylic solvent and removing solvent by vacuum drying. This process suffers from disadvantages such as highly specialized equipment, longer drying time, residual solvents in the product and need to recovery of solvents.

U.S. Pat. No. 6,274,740 describes the method for conversion of crystalline form of Atorvastatin calcium to amorphous form which comprises of using two solvents. The process involves complete removal of solvent under high vacuum (5 mm of Hg) at high temperature (90° C.) using capital intensive equipment and further vacuum drying at high temperature leads to degradation of the product.

U.S. Pat. No. 6,528,660 describes preparation of amorphous Atorvastatin calcium by solvent precipitation. Atorvastatin calcium is dissolved in tetrahydrofuran and an anti solvent is added to obtain amorphous material. However, this process makes use of large quantities of solvents thus requiring efficient solvent recovery. A further drawback of this process is the amount of residual solvents left even after extensive drying.

PCT Application WO 01/28999 A1 describes an alternate process wherein amorphous Atorvastatin calcium was prepared by crystallization from a lower alkanol containing 2-4 carbon atoms.

US application 2002/0183527 also describes the preparation of amorphous Atorvastatin calcium by solvent precipitation. Crystalline Atorvastatin calcium is dissolved in alcohol and ether is added to precipitate the amorphous product.

PCT Application WO 03/78379 discloses the preparation of amorphous Atorvastatin calcium having particle size 1 to 150 microns by dissolving atorvastatin calcium in a hydroxylic solvent and removing the solvent by freeze drying or by spray drying and subsequently subjecting the product to milling.

PCT Application WO 02/83637 discloses the preparation of amorphous Atorvastatin calcium directly from diol protected tert-butyl ester. But disadvantage of this process is that amorphous Atorvastatin calcium prepared is contaminated with unreacted diol compound and needs purification.

PCT Application WO 03/018547 describes a process for the preparation of amorphous Atorvastatin calcium which comprises hydrolyzing the lactone form of Atorvastatin with aqueous base, extracting with organic solvent and adding the same to an anti-solvent to precipitate the product and finally filtering the product to afford amorphous Atorvastatin calcium. Alternatively, amorphous Atorvastatin calcium is prepared from crystalline Atorvastatin calcium, which comprises dissolving the crystalline Atorvastatin calcium in an organic solvent, distilling off half of the solvent and adding the same to an anti solvent to precipitate the amorphous product.

Thus, it is evident that most of the methods described in the prior art to prepare amorphous Atorvastatin calcium have some disadvantages such as involving use of large volumes of solvents, thus requiring efficient solvent recovery, low yields and high limits of residual solvents even after sufficient drying which makes these processes industrially unattractive from economical and handling point of view.

It is therefore an object of the present invention to provide a process for the preparation of amorphous Atorvastatin calcium which avoids production of mixture of amorphous and crystalline forms and which is economical and capable of being practiced on a commercial scale.

According to the present invention amorphous Atorvastatin calcium is prepared by spray drying or pulverization of crystalline atorvastatin calcium or a mixture of crystalline and amorphous forms of atorvastatin calcium.

### SUMMARY OF THE INVENTION

Accordingly, the present invention provides a process for producing amorphous Atorvastatin calcium, which comprises the steps of:

dissolving a mixture of crystalline and amorphous atorvastatin calcium of Formula I in any hydroxylic solvent such as methanol, ethanol, isopropanol and their aqueous mixtures thereof,

subjecting the resulting solution to spray drying at a temperature of 40° C. to 90° C. to remove the solvent and

isolating the amorphous Atorvastatin calcium in pure form.

In one of the embodiment, the present invention provides a process for producing amorphous Atorvastatin calcium, which comprises the steps of:

dissolving any crystalline form of Atorvastatin calcium of Formula I in any hydroxylic solvent such as methanol, ethanol, isopropanol and their aqueous mixtures thereof,

subjecting the resulting solution to spray drying at a temperature of 40° C. to 90° C. to remove the solvent and

isolating the amorphous Atorvastatin calcium in pure form.

In another embodiment, the present invention provides a process for producing pure amorphous Atorvastatin calcium, which comprises the steps of:

subjecting the crystalline Atorvastatin calcium to pulverization at a temperature of 20-25° C., without using any solvent until the said crystalline Atorvastatin calcium is converted to amorphous form,

isolating the amorphous Atorvastatin calcium.

### BRIEF DESCRIPTION OF ACCOMPANYING DRAWINGS

FIG. 1: X-ray powder diffractogram of amorphous Atorvastatin calcium prepared by spray drying (Example 1)

Vertical axis: Intensity (CPS)

Horizontal axis: Two theta (degrees)

FIG. 2: X-ray powder diffractogram of amorphous Atorvastatin calcium prepared by spray drying (Example 2)

Vertical axis: Intensity (CPS)

Horizontal axis: Two theta (degrees)

FIG. 3: X-ray powder diffractogram of amorphous Atorvastatin calcium prepared by pulverization. (Example 3)

Vertical axis: Intensity (CPS);

Horizontal axis: Two theta (degrees)

FIG. 4: X-ray powder diffractogram of crystalline Atorvastatin calcium

Vertical axis: Intensity (CPS)

Horizontal axis: Two theta (degrees)

FIG. 5: Infrared spectrum of amorphous Atorvastatin calcium prepared by spray drying (Example 1)

FIG. 6: Infrared spectrum of amorphous Atorvastatin calcium prepared by spray drying (Example 2)

FIG. 7: Infrared spectrum of amorphous Atorvastatin calcium prepared by pulverization. (Example 3)

FIG. 8: Infrared spectrum of crystalline Atorvastatin calcium

### DETAILED DESCRIPTION OF THE INVENTION

The instant invention relates to simple and efficient process for the preparation of amorphous Atorvastatin calcium, which involves the conversion of crystalline atorvastatin calcium or a mixture of crystalline and amorphous atorvastatin calcium by spray drying using hydroxylic solvents or by pulverization.

The Atorvastatin calcium may be prepared by the methods described in the previously mentioned US patents, which are incorporated herein by reference. The crystalline Atorvastatin calcium may be prepared by the methods described in U.S. Pat. No. 5,969,156.

In one of the embodiment of the present invention the preparation of amorphous Atorvastatin calcium is carried out easily by spray drying a solution of crystalline atorvastatin calcium or a mixture of crystalline and amorphous forms of atorvastatin calcium in any suitable hydroxylic solvent. The hydroxylic solvent can be selected from group of solvents such as methanol, ethanol, isopropanol and like and their aqueous mixtures thereof.

We have found that Atorvastatin calcium is heat stable and withstands spray drying conditions. Spray drying system can be operated in known manner to obtain amorphous Atorvastatin calcium essentially free from crystalline material. This technique is safe and economic due to high recovery of product and use of a single solvent to prepare amorphous Atorvastatin calcium. The product obtained from spray drying has the form of hollow microspheres which can be conveniently compounded into pharmaceutical preparation. Residual solvent may be present in the product, immediately after spray drying which can be reduced by further drying under reduced pressure. Typically the crystalline Atorvastatin calcium is dissolved in any hydroxylic solvent to obtain 5-30% w/v solution, but preferably 10-15% w/v. The solvent may be heated and small quantities of water may be added if required to dissolve the material. The solution is then introduced in mini spray dryer manufactured by Buchi, Switzerland using nitrogen gas as drying gas. The gas inlet temperature is adjusted in the range from 50° C. to 90° C. and gas outlet temperature is adjusted between 40° C. to 80° C. The product is spray dried until white, amorphous powder is obtained, which is further dried under reduced pressure. During spray drying no degradation takes place and product obtained is having residual solvent less than 500 ppm.

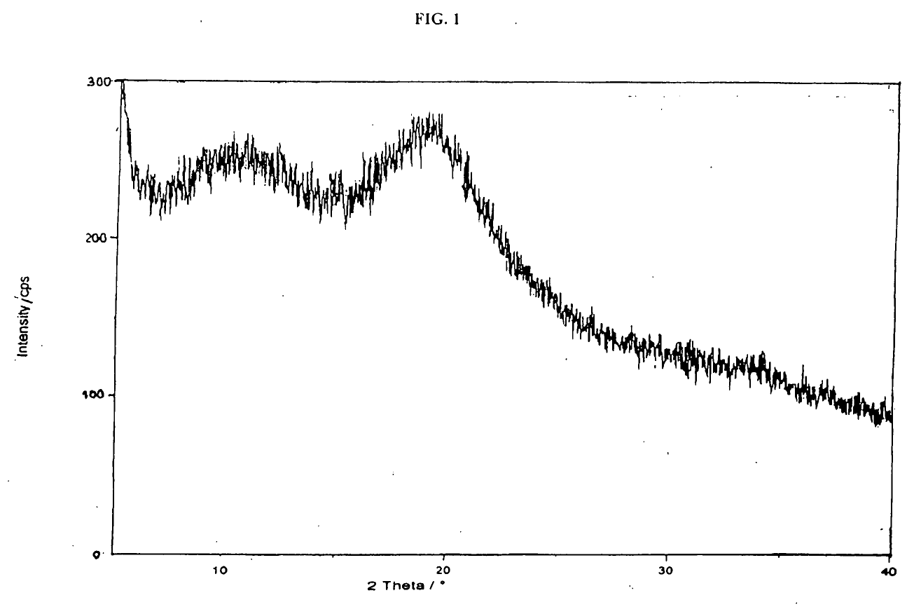

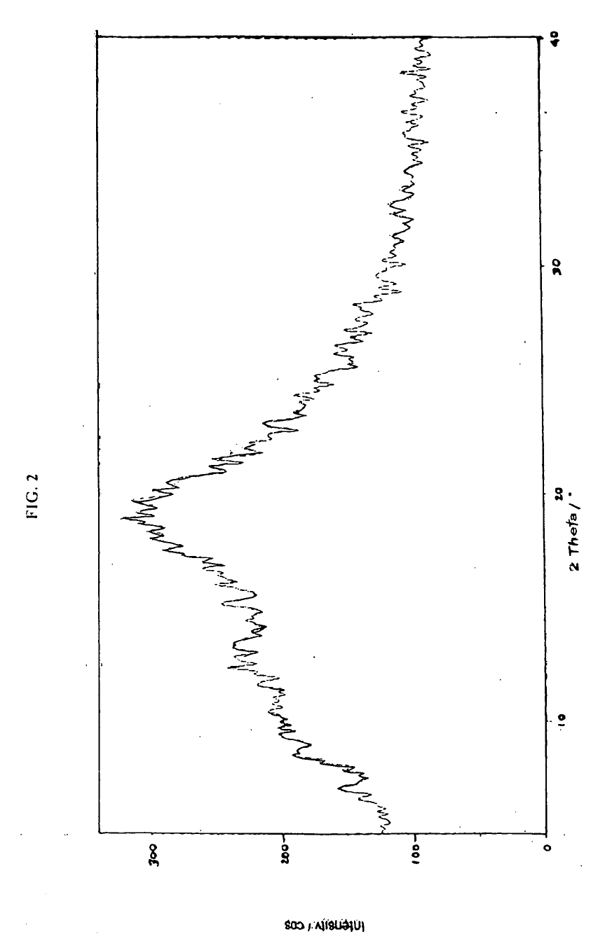

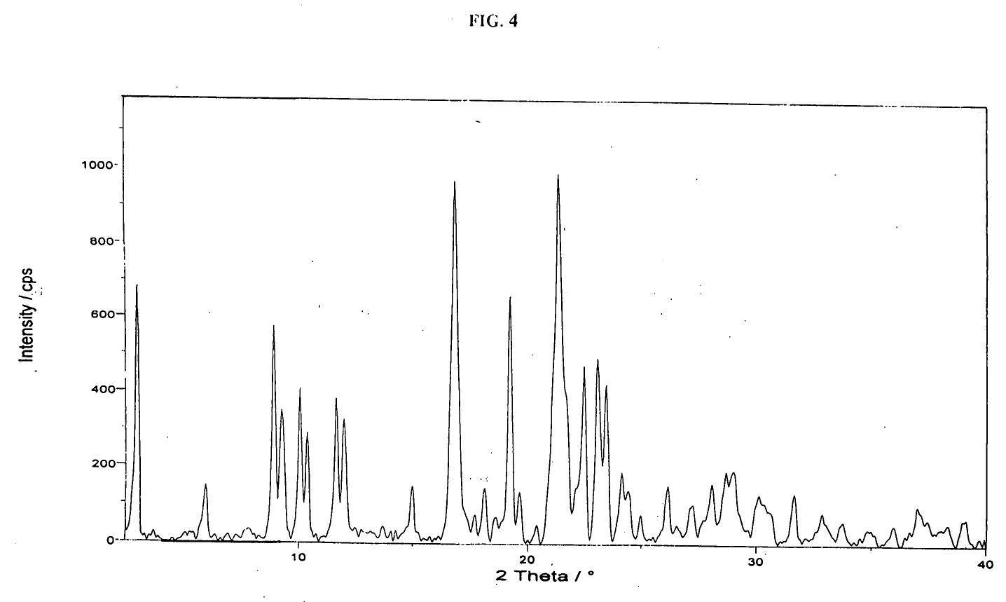

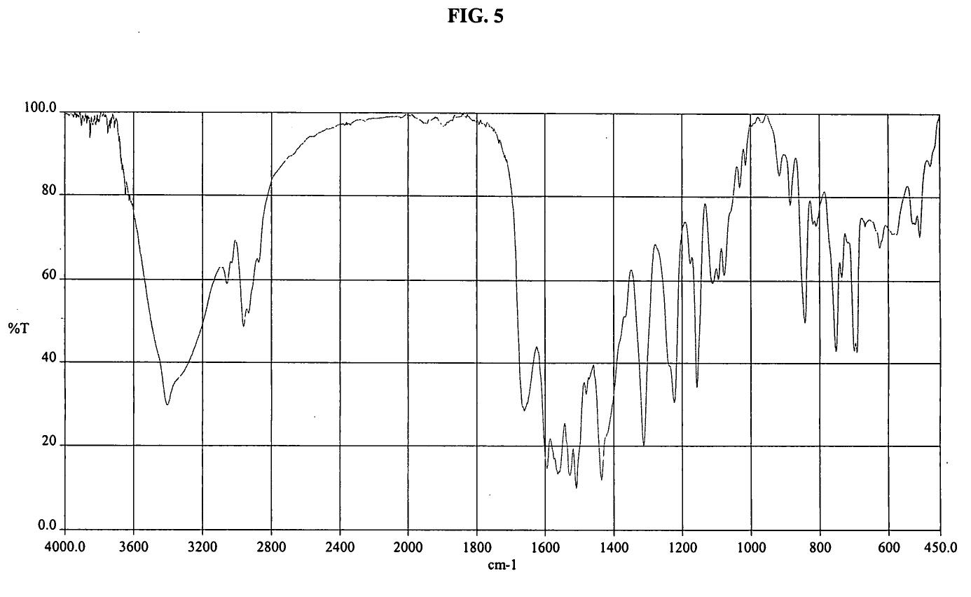

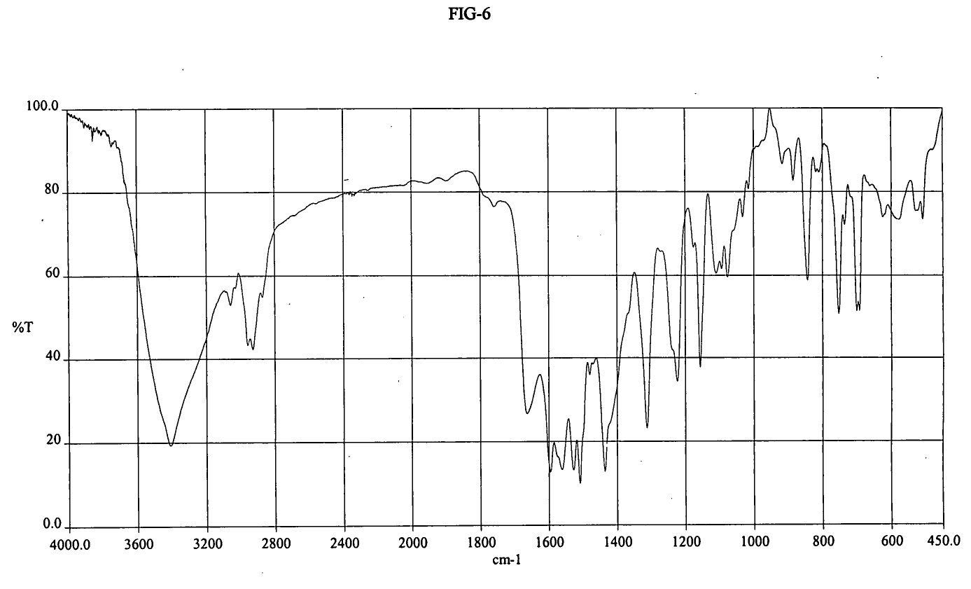

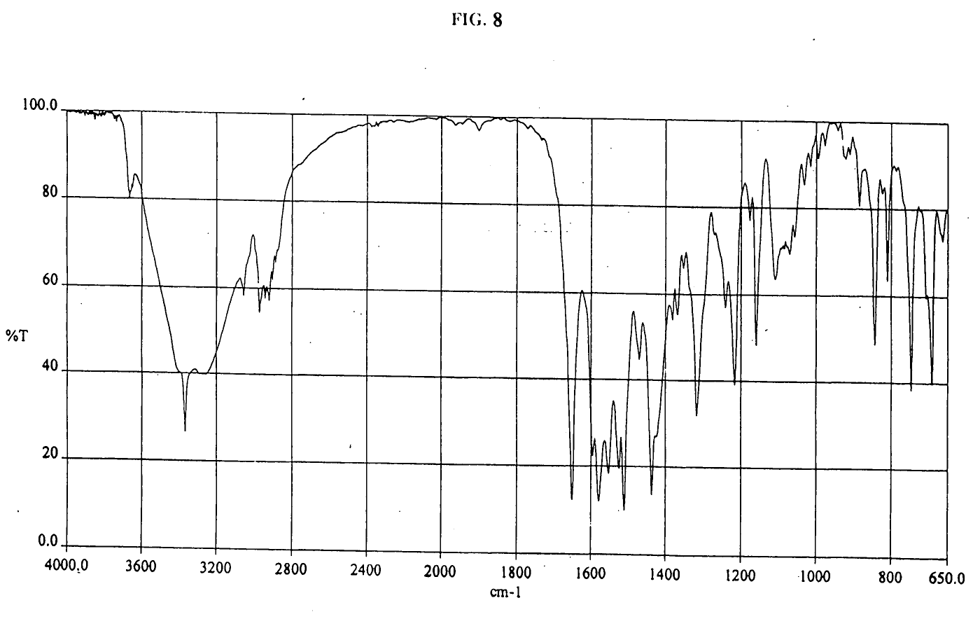

Amorphous Atorvastatin calcium, prepared by the spray drying of the crystalline form has been characterized by powdered X-ray diffraction and infrared spectra. Thus, powdered X-ray diffraction of amorphous and crystalline Atorvastatin calcium were determined on Seifert XRD 3003TT system using a copper target X-ray tube, a nickel filter and sample was placed in a Pyrex glass holder. The powdered X-ray diffractograms of amorphous Atorvastatin calcium show no peaks (FIG. 1 and FIG. 2) thereby indicating that product is amorphous, whereas FIG. 4 is a characteristic powdered X-ray diffractogram of crystalline form of Atorvastatin calcium. The infrared absorption spectra of amorphous and crystalline forms of Atorvastatin calcium were determined on Perkin Elmer-spectrum ONE infrared spectrophotometer. The infrared spectra of amorphous Atorvastatin calcium also shows difference with infrared spectrum of crystalline Atorvastatin calcium (FIG. 5, FIG. 6 and FIG. 8).

In another embodiment of the present invention the preparation of amorphous Atorvastatin calcium is carried out by pulverization. Here, pulverization means crushing or grinding of a substance to fine particles.

Specifically, the pulverization of crystalline form of Atorvastatin calcium was carried out by using mortar and pestle or mechanical grinding. The said process is conducted in neat without using any solvent and is amenable to large scale production. Typically the conversion of crystalline form to amorphous form is completed in 8-10 hrs at ambient temperature and this can be monitored by infrared and X-ray powder diffraction analyses during crushing operation itself.

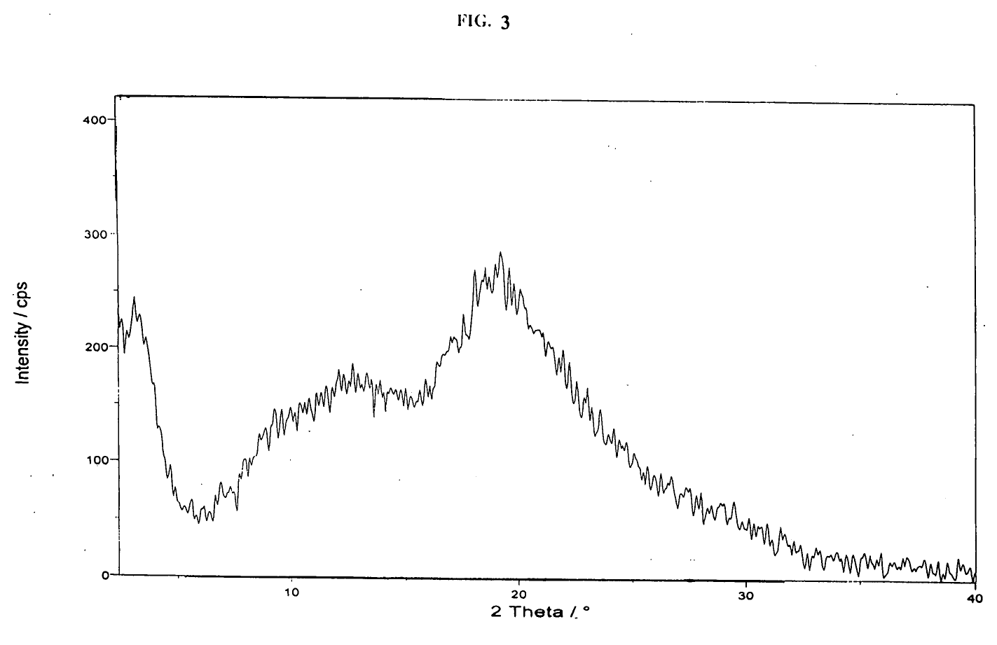

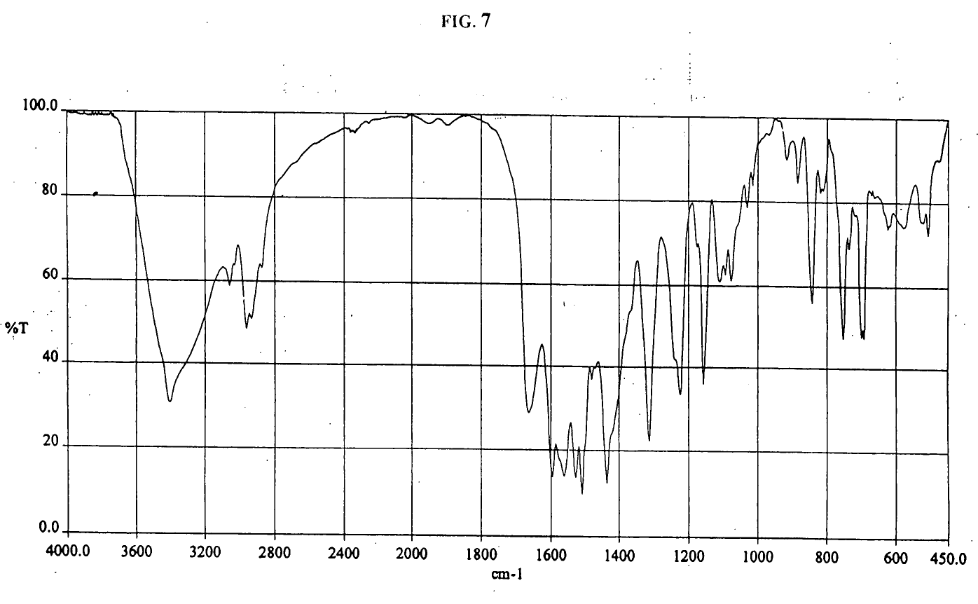

Amorphous Atorvastatin calcium, prepared by pulverizing the crystalline form has been characterized by X-ray powder diffraction, which is identical with the reported and shown in FIG. 3. The X-ray powder diffractogram (FIG. 3) of amorphous Atorvastatin calcium shows that peaks characteristic of crystalline form of Atorvastatin calcium (FIG. 4) are entirely absent. The infrared spectrum of amorphous Atorvastatin calcium also shows difference from the infrared spectrum of crystalline Form-I of Atorvastatin calcium (Refer FIG. 7 and FIG. 8).

Thus the major advantages of the present invention as compared to prior art processes are high purity, cost effectiveness, absence of residual solvent impurity, consistency and hence commercially attractive. Any form of crystalline atorvastatin calcium or a mixture of crystalline and amorphous atorvastatin calcium that can be dissolved in the said solvents can be spray dried to obtain amorphous Atorvastatin calcium.

In pulverization method, no solvent is required to isolate the product and hence no mother liquor is obtained which is difficult to regenerate.

Further details of the present invention are to be found in the following examples without limiting it:

### EXAMPLE 1

Preparation of Amorphous Atorvastatin Calcium

15 g of crystalline Atorvastatin calcium was dissolved in 100 ml of methanol. The clear solution was then introduced into spray dryer using nitrogen gas as drying gas. The product was then spray dried at a temperature of 58° C. to 62° C. until a white amorphous powder of Atorvastatin calcium was obtained. The amorphous material, thus obtained was dried under reduced pressure till LOD ≦0.5% w/w, having residual solvent level of 0.015% w/w for methanol. Purity 98.7% by HPLC

### EXAMPLE 2

Preparation of Amorphous Atorvastatin Calcium

10 g of crystalline Atorvastatin calcium was dissolved in 200 ml of ethanol and heated to 60° C. to get clear solution. The clear solution was then spray dried at 80° C. to 85° C. until a white amorphous powder of Atorvastatin calcium was obtained. The amorphous material thus obtained was dried under reduced pressure till moisture content ≦1% w/w, having residual solvent level of 0.009% w/w for methanol and ethanol was not detected. Purity 98.5% by HPLC.

### EXAMPLE 3

Preparation of Amorphous Atorvastatin Calcium

Crystalline Atorvastatin calcium (Form I, 10 g) was taken in mortar. It was then pulverized using mortar and pestle for about 8-9 hours at room temperature. Atorvastatin calcium (9.9 g) thus obtained was characterized by powdered X-ray diffraction pattern (FIG. 1) showing no peaks, thus demonstrating that the nature of product is amorphous.

### EXAMPLE 4

Preparation of Amorphous Atorvastatin Calcium

Crystalline Atorvastatin calcium (10 g) was taken. The solid mass was then pulverized using mechanical grinder for about 8-9 hours at room temperature. The powdered X-ray diffraction pattern indicated that the material obtained is amorphous Atorvastatin calcium.

We claim:

1. A process for producing amorphous Atorvastatin calcium, which comprises the steps of:
dissolving a mixture of crystalline and amorphous Atorvastatin calcium of Formula I 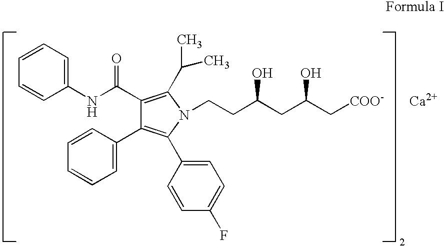
in a hydroxylic solvent,
drying the resulting solution to remove the solvent,
and isolating the amorphous Atorvastatin calcium in pure form.

2. The process according to claim 1 wherein the said solvent is removed by spray drying.

3. The process according to claim 2 wherein the spray drying is carried out at a temperature between 40° C. and 90° C.

4. The process according to claim 1 wherein the said hydroxylic solvent is selected from the group consisting of methanol, ethanol, isopropanol, a mixture thereof, and a mixture of at least one of the afore mentioned hydroxylic solvent and water.

5. The process according to claim 1, wherein highly crystalline Atorvastatin calcium is used in the said dissolving step in place of a mixture of crystalline and amorphous Atorvastatin calcium.

6. The process according to claim 1 wherein the said pure amorphous Atorvastatin calcium obtained is substantially free from residual solvents.

7. A process for the preparation of pure amorphous Atorvastatin calcium comprising of pulverization of highly crystalline Atorvastatin calcium in solid form and without any added solvent, until the said crystalline Atorvastatin calcium is converted to amorphous Atorvastatin calcium.

8. The process according to claim 7 wherein the said pulverization is carried out at a temperature between 20° C. and 40° C.

9. The process according to claim 7 wherein said pulverization is carried out using a mechanical grinder.

10. The process according to claim 7 wherein the mortar and pestle is used for carrying out the said pulverization.

11. The process according to claim 7 wherein the process of conversion of crystalline Atorvastatin calcium to said amorphous Atorvastatin calcium is completed in 1 to 10 hours.
